(19) United States
(12) Patent Application Publication
(10) Pub. No.: US 2017/0348235 A1
(43) Pub. Date: Dec. 7, 2017

(54) MULTI-PHASE RELEASE OF SPORTS NUTRITION AND ENERGY DRINK COMPOSITIONS UTILIZING LIPID PARTICULATES

(71) Applicant: Corr-Jensen, Inc., Denver, CO (US)

(72) Inventor: Tyler O. White, Greenwood, SC (US)

(21) Appl. No.: 15/684,471

(22) Filed: Aug. 23, 2017

Related U.S. Application Data
(63) Continuation-in-part of application No. 15/265,662, filed on Sep. 14, 2016.
(60) Provisional application No. 62/219,283, filed on Sep. 16, 2015.

(51) Int. Cl. A61K 9/14; A61K 9/00; A61K 31/198; A23L 2/52; A61K 31/661; A61K 31/522

(52) U.S. Cl. CPC A61K 9/145; A61K 31/661; A61K 31/522; A61K 31/198; A23L 2/52; A61K 9/0095; A61K 9/146; A23V 2002/00

(57) ABSTRACT

Multi-phase release compositions, methods of using the compositions, and methods for manufacturing the compositions are provided. Multi-phase release compositions include ingredients useful in sports nutrition including ingredients useful in muscle building, energy output and weight loss. Sports nutrition ingredients are designed for immediate and extended release dependent on the particular sport nutrition motivated use. One sports nutrition ingredient that can be utilized in either or both the immediate release phase or the extended release phase is phosphatidic acid.

### RELATED APPLICATIONS

This application is a Continuation-In-Part of U.S. patent application Ser. No. 15/265,662, entitled “MULTI-PHASE RELEASE OF SPORTS NUTRITION AND ENERGY DRINK COMPOSITIONS UTILIZING LIPID PARTICULATE,” filed Sep. 14, 2016, which claims priority under 35 U.S.C. 119 (e) to U.S. Provisional Patent Application Ser. No. 62/219,283, entitled “MULTI-PHASE RELEASE OF SPORTS NUTRITION AND ENERGY DRINK COMPOSITIONS UTILIZING LIPID PARTICULATE,” filed Sep. 16, 2015, the disclosure of which is hereby incorporated by reference in its entirety.

### TECHNICAL FIELD

The present invention is directed to sports nutrition and energy drink compositions and to methods of manufacturing and using the same.

### BACKGROUND OF THE INVENTION

Sports nutrition and energy supplements continue to have expanding markets. Typical supplements, which for purposes herein includes, but not limited to, energy drinks, are designed to facilitate one or more aspects of a user's overall health including muscle growth, energy output, energy sustainability and/or weight loss.

Sport supplements can include, for example, one or more of proteins, amino acids, performance enhancers, hormones, fat burners, and omega-3 fats. Typical supplements are designed for oral intake and dissolution in the stomach for immediate availability to the subject. Although most supplements can be taken at any time, maximum benefit is created by taking the supplement at a time when the supplement constituents are needed for an intended use. For example, a supplement high in casein protein is typically ingested after a work-out due to its high level of tyrosine and proline, building blocks in muscle recovery.

However, sports nutrition supplements are crudely designed to facilitate the availability of constituents for an intended use. Conventional sports nutritional supplements provide little or no control over availability of the constituents to the user beyond the timing of ingestion for a desired benefit, i.e., take the supplement between meals, just prior to a work-out, during a work-out, etc. The control of a supplement's release in a user is lost once the supplement is ingested.

The present invention is directed toward overcoming one or more of the problems discussed above.

### BRIEF DESCRIPTION OF THE DRAWINGS

FIG. 1 shows a phosphatidic acid release profile in accordance with aspects of the present invention.

FIG. 2 shows a phosphatidic acid release profile in accordance with aspects of the present invention.

FIG. 3 shows phosphatidic acid release profile under various specific dissolution conditions, showing specificity of release is based on lipid compositions.

### SUMMARY OF THE INVENTION

Disclosed herein are multi-phase active ingredient release compositions, methods of using these multi-phase release compositions and methods of manufacturing the same. In typical embodiments the active ingredient(s) are useful as sports nutrition supplements, including energy drinks or enhancers, and provide an added and unexpected benefit to the user.

Embodiments include multi-phase release compositions having one or more active ingredients where the one or more active ingredient is designed to be released in a user over an extended period of time. In some aspects, the extended release phase composition may include an immediate release phase. In other aspects, the multi-phase release composition includes two or more different or overlapping extended release phases with or without an immediate release phase.

In typical embodiments, an immediate release phase comprises one or more active ingredients for release upon ingestion of the multi-phase release composition by the user. Active ingredients in the immediate release phase are released based on the user's normal digestive processes and more typically the user's normal upper gastrointestinal tract processes. Active ingredients useful in inclusion in the immediate release phase include, but not limited to, caffeine, phosphatidic acid, creatine, amino acids, yohimbine, synephrine, theacrine, vitamins, and the like. In some aspects the immediate release phase is a powder, and in other aspects the immediate release phase is composed of active ingredient(s) dissolved in an aqueous liquid.

In typical embodiments, an extended release phase comprises one or more active ingredients carried or coated on one or more lipid particulates, the extended release phase being designed to prolong, delay or target release of an active ingredient to the user's bloodstream. Coated or carried active ingredients on lipid particulates in the extended release phase are designed to be released over any period of time beyond what would be expected from the same active ingredient in the absence of the lipid particulate, i.e., by itself. Active ingredients useful in inclusion in the extended release phase include, but not limited to, caffeine, phosphatidic acid, creatine, amino acids, yohimbine, synephrine, theacrine, vitamins, and the like. Lipids useful in forming the extended release phase include, but not limited to, stearic acid, stearyl alcohol, glycerol behenate, gelucires, monoglycerides, diglycerides, triglycerides, and the like. Typical lipids for use herein are solid at room and storage temperatures. Note that the active ingredient phosphatidic acid (a phospholipid) can also be used as a lipid to modify the extended release, in a sense, it can act alone, or in combination with other lipids, to effect its own extended release.

In still other embodiments, an extended release phase comprises one or more active ingredients carried or coated on a combination of one or more lipids and/or one or more waxes. The lipid and wax particulate(s) being designed to prolong, delay or target release of an active ingredient to the user's bloodstream. The wax addition to the particulate may also modify the melting point of the particulate and provide for additional storage stability of the particulate. Active ingredients useful in inclusion in this extended release phase include, but not limited to, caffeine, phosphatidic acid, creatine, and the like. Waxes useful in forming this extended phase include, but not limited to, bees wax, rice bran wax, camauba wax, candelilla wax, glycerol monostearate, glycerol oleate, spermaceti, and the like. Note that it is envisioned that the particulate could include only the wax and active ingredient component, but would more typically be present at 1% to 50% w/w with the lipid component.

As noted above, the present disclosure may include multi-phase release compositions having two or more extended release phases, each extended release phase designed to prolong, delay or target the release of one or more active ingredients upon ingestion by a user. Multi-phase release compositions including two or more extended release phases are proposed by modifying the amount and/or composition of lipid and/or wax associated with the active ingredient. For example, an extended release phase may include a first lipid composition coated with an active ingredient and a second lipid and wax composition coated with the same or different active ingredient. The first and second compositions designed to release active ingredients over different periods of time.

As such, aspects herein include multi-phase release compositions having multiple extended release phases, each phase designed to extend the release of one or more active ingredients in a user such that a first phase could release a first active ingredient over 60 minutes, for example and a second active ingredient over 120 minutes, also for example. As can be imagined, various combinations of immediate and multiple extended release phases can be combined to maximize a compositions' utility to a user.

In some aspects, the present disclosure presents multi-phase release compositions having the same active ingredient(s) in the immediate release phase as in the extended release phase. In other aspects, the present disclosure presents multi-phase release compositions having different active ingredient(s) in the immediate release phase from the extended release phase, for example, a multi-phase release composition having phosphatidic acid in the immediate release phase and caffeine in the extended release phase. As can be envisioned, aspects of the disclosure also include a plurality of active ingredients where some ingredients are found only in one or the other phase but other active ingredients are found in both phases. For example, an immediate release phase that includes caffeine and phosphatidic acid and an extended release phase having caffeine, creatine, and phosphatidic acid.

### DESCRIPTION

Embodiments herein provide multi-phase sports nutritional release compositions, methods of using these compositions, and methods of manufacturing the same. Multi-phase sports nutritional release compositions are for oral administration and typically include, but not limited to, at least one extended release phase, and more typically includes at least one extended release phase paired with an immediate release phase. Some multi-phase sports nutritional release compositions in accordance with embodiments herein include two or more extended release phases paired with an immediate release phase.

The immediate release phase is usually composed of one or more active components or ingredients useful in sports nutrition and energy drinks. For simplicity sake the term “sports nutrition” will refer throughout the disclosure to ingredients that include energy enhancement and maintenance. Active ingredients useful in the immediate release phase are released to a user's bloodstream based on the user's normal digestive timing.

The extended release phase can provide a sustained release, a delayed release or a pre-determined targeted timed release of sports nutrition ingredients. The sports nutrition ingredients can be the same or different between the immediate and extended release phases. The delivery of an extended release phase component to a user provides a significant advantage over traditional sports nutrition compositions and the simultaneous delivery of at least one immediate release phase component and at least one extended release phase component provides a significant advantage in the utility of a sports nutritional composition.

Multi-phase sports nutritional release compositions (referred to herein as “release compositions”) of the present disclosure may confer both immediate and prolonged absorption of an active component(s) in the user's gastrointestinal tract and more typically in the upper parts of the user's gastrointestinal tract. Prolonged, delayed and/or targeted release of an active component is coordinated with the active compounds utility thereby facilitating and extending that utility. In typical embodiments, a release composition is designed to provide immediate and extended release of one or more of the same or different active components, or a combination thereof. In one embodiment, the active component is phosphatidic acid. In some embodiments the phosphatidic acid is found in both the immediate and extended release phases. In another embodiment, the active component is caffeine. In these embodiments, the caffeine may be found in either or both the immediate and extended release phases.

Other active ingredients can be used for delivery by compositions described herein. For purposes herein, an active ingredient or component is one or more various active ingredients within the sports nutrition art including but not limited to: stimulants (caffeine, synephrine, yohimbine, theobromine, etc.), botanical extracts (yohimbe bark, ashwagandha, citrus aurantium, coleus forskohlii, salvia sclarea, rauwolscine, ginseng, black pepper extract, theacrine, etc.), muscle/strength compounds (creatine (various formats), phosphatidic acid, HMB, HILA, diosgenin), amino acids (natural and synthetic, including Leucine, Isoleucine and Valine), vasodilation/pump compounds (citrulline and various formats, various forms of nitrate, norvaline, pycnogenol, arginine and related compounds (ex: arginine-silicate inositol), GPLC, etc.), vitamins/minerals (ascorbic acid, thiamin, niacin, vitamin B6, riboflavin, iodine, zinc, and the like), and testosterone/libido supporting compounds (tribulus terrestris, fenugreek, epimedium, eurycoma longifolia, maca, avena sativa, etc.), or a combination thereof. Any useful combination of active ingredients can be found in either or both the immediate and extended release phases.

In one embodiment, the immediate release phase is a dry bulk powder that includes one or more active ingredients. Bulk powders in accordance with the disclosure are typically homogenous and composed of fine to very fine particles, which will flow freely when shaken or tilted. Particulate size can vary dependent upon the active ingredient and the intended use, but typically are of a diameter of between about 50 μm to about 1,500 μm and more typically between about 50 μm and about 1,200 μm. Illustrative particle sizes include: D₁₀, D₅₀, D₉₀ and D[4,3].

In an alternative embodiment, the immediate release phase is an aqueous liquid having one or more soluble active ingredients therein. Aqueous liquids can have any commonly used pH parameters known in the nutritional and/or energy drink arts. In some embodiments, the aqueous liquids have a pH from about 2.0 and 5.0, and in other embodiments from about 3.0 to about 4.0 or from about 3.4 to about 3.9.

A typical immediate release phase has one, or at least: two, three, four, five, six, seven, eight, nine, ten, eleven, twelve, and the like, sports nutrition ingredients or components. As noted above, immediate release phase embodiments are typically a dry bulk powder or an aqueous liquid. Immediate release phases may also include non-sports nutrition ingredients, including flavoring, preservatives, fillers, suspension aids, coloring agents, specific gravity adjustment agents, pH adjustment agents, and the like.

For dry bulk powder embodiments, weight percent, of each active component is modified for the particular use of the sports nutrition compositions. Additional non-active ingredients can be included in the immediate release phase powder including but not limited to binders, fillers, sugars, flavoring agents, coloring agents, antioxidants, preservatives and the like.

For aqueous liquid embodiments, grams/liter of each active component is modified for the particular use of the sports nutritional composition. Typical embodiments include up to an amount of an active ingredient that is still soluble in the aqueous liquid. Additional non-active ingredients in the aqueous liquid can include but not limited to: soluble fiber(s), pH adjusting agents, sugars, flavoring agents, coloring agents, antioxidants, preservatives, and the like.

In one embodiment, the immediate release phase is a dry bulk powder or liquid including about 1-15% anhydrous caffeine by weight, and more typically, 10-15% anhydrous caffeine by weight. In another embodiment, the immediate release phase is a dry bulk powder or liquid including about 1-15% amino acids, e.g., Leucine, Isoleucine, Valine, etc. or combination thereof, by weight, and more typically, 10-15% amino acids by weight. In some embodiments, caffeine and amino acids can be combined to provide the above described amounts.

In another embodiment, the immediate release phase is a dry bulk powder including a combination of caffeine, beta-alanine, creatine, betaine, citrulline malate, n-acetyl-1-tyrosine, niacin, black pepper extract, vitamin B-12, and alpha yohimbine.

In yet another embodiment, the immediate release phase is a dry bulk powder including a combination of phosphatidic acid or phosphatidic acid and at least one other active ingredient. One illustrative phosphatidic acid combination includes beta-alanine (Carnosyn®), Betaine Anhydrous (Trimethyl Glycine), L-Citrulline DL-Malate 2:1, Creatine HCL, N-Acetyl L-Tyrosine, Caffeine Anhydrous, Niacinamide/Nicotinamide Vitamin B3, Bioperine (Piper Nigrum), KSM-66, Beverage Grade, Leucine, Instantized, and Mediator 50 P 50% PA.

In yet another embodiment, the immediate release phase is an aqueous liquid including but not limited to Caffeine, Taurine, Ginseng, Vitamin B-6, Vitamin B-12, Citicoline, Tyrosine, N-Acetyl-L-Tyrosine, Glucuronolactone, Guarana Extract, L-Carnitine, Niacin or a combination thereof, each at levels to provide the nutritional intended effect.

For purposes herein, an extended release phase in accordance with one embodiment of the disclosure is composed of one or more functional lipids having a relatively high melt point, where the lipids have a solid state at use and storage temperatures, in combination with one or more active ingredients (the ingredient(s) that will be released over time). Lipids for use herein include at least: stearate, saturated monoglycerides, diglycerides, triglycerides (glycerol behenate, glycerol dibehenate, Compritol™, glycerol monostearate), and the like. Lipids for use herein can also be any combination of the above including any two, three, four, and the like lipid for any particular use. An extended release phase is designed using lipid combinations to provide a target release profile for enhancing an active ingredient's use capabilities. In this way, the extended release phase can be a sustained release, a delayed release or a targeted release for any one or more active ingredients. Release profiles in accordance with embodiments herein can facilitate active ingredient release of up to about 15 minutes, up to 30 minutes, up to 45 minutes, up to 60 minutes, up to 90 minutes, up to 2 hours, 3 hours, 4 hours, 5 hours, 6 hours, 9 hours or 12 hours.

As noted above, typical embodiments have lipid particulates with a melt onset above room temperature, and more typically above 40° C. In other embodiments, lipid particulates are designed to have melt onset at about 50° C., at about 60° C. and at about 70° C. Melt onset stability of the lipid particulate is helpful in both manufacturing of the release composition but also in stability of the composition for use and storage.

An extended release phase in accordance with another embodiment of the disclosure is composed of one or more functional lipids having a relatively high melt point in combination with one or more waxes having a relatively high melt point. The term wax is intended to include, but not be limited to, bees wax, rice bran wax, camauba wax, candelilla wax, glycerol monostearate, glycerol oleate, spermaceti, and the like. An extended release phase in this embodiment is designed using a lipid and wax combination to provide a target release profile for enhancing an active ingredient's use capabilities. In some aspects, the extended release phase may include only one or more waxes and one or more active ingredients, if the combination provides a necessitated release profile. However, where both a lipid and wax is included in the extended release profile, the wax is typically at a 1% to 50% w/w combination with the lipid. In other embodiments, the wax is at a 5% to 40% w/w combination with the lipid, and in still other embodiments, the wax is at a 10% to 30% w/w combination with the lipid.

Particulates for use in the extended release phase, and in accordance with embodiments herein, do not interfere with the mechanism of action of the active ingredient(s). While not bound by a theory, it is believed that the lipid and/or wax component of a particulate forms a matrix from which the active ingredient is released. The release time of the active ingredient can be varied by changing amounts, types, and combinations of the lipids and waxes used to form the extended release phase. In addition, and as discussed below, the manner of manufacture and form of the extended release phase also provides an effect on the release time of the active ingredient.

Additional matrix forming components may be added to the lipid and/or wax particulates as well, as is needed, including: succinic acid, citric acid, malic acid, lactose, sucrose, dextrose, xylitol, glyceryl behenate, lactic acid, hypromellose, hydrophilic cellulose, polyethylene glycols and the like.

Particulates for use in the extended release phase, and in accordance with embodiments herein, can be processed through several manufacturing methods including: extrusion, melt spray congealed, solid lipid pellet technology, granulation, and the like. Proper combinations of lipid, wax, or lipid and wax, are added or combined with the active ingredient(s) to provide a particular extended release phase for that active ingredient(s). Lipid, wax or lipid/wax carry or encase active ingredients, and the properties of the matrix are designed to achieve an extended dissolution within the gastrointestinal environment in a user.

An extended release phase is composed of the appropriate lipid, wax or lipid/wax particulate(s) and active ingredient(s) to provide the active ingredient(s) release profile desired for the release composition. The composition of the particles can be modified to fit the particular active release profile. The content of any one lipid, wax or lipid/wax particulate to active ingredient can also be used to modify an active ingredients release profile. To prepare the extended release phase, an amount of one or more active ingredients is combined directly with an amount of one or more target lipid, wax, lipid/wax particulates to make active ingredient carrier or coated particulates/matrix.

The active ingredients carried by the lipid, wax or lipid/wax particulates will be released over a period of time dependent upon the composition and amounts of the lipid and/or wax particulates as compared to the amounts of carried/coated active ingredient. In one embodiment, the active ingredient carried on the particulate is phosphatidic acid. In other embodiments, the active ingredient carried on the lipid and/or wax particulate is phosphatidic acid in combination with one or more additional active ingredients, e.g., caffeine, amino acids, etc. It is noted that any active ingredient useful in the sport nutrition art, and as described herein, can be carried alone or in combination on the lipid and/or wax particulates to provide an extended release phase.

Illustrative embodiments of extended release phase coated particulates include: 45% by weight (wt %) stearic acid, 45 wt % phosphatidic acid, 7 wt. % titanium dioxide and 3 wt % FD&C Yellow Lake No. 5; 50 wt % stearic acid and 50 wt % phosphatidic acid; 50 wt % Compritol® and 50 wt % phosphatidic acid; 45 wt % Compritol®, 5% Pluronic® and 50% phosphatidic acid; and 25 wt % stearic acid, 25 wt % Compritol® and 50 wt % phosphatidic acid.

In one embodiment, designed lipid and/or wax particulates loaded with desired active ingredients are then blended with a dry powder immediate release phase to form a targeted release composition, i.e., a multi-phase release composition.

Where the immediate release ingredients are in a liquid form, a percentage of the extended release phase may release some of its active ingredient into the immediate release phase. Where the active ingredient on the extended release phase is only meant for extended release, the extended release phase can be added to the immediate release phase just prior to ingestion by the user, or the active ingredient coated lipid and/or wax particulate can be further encapsulated in a material that only dissolves and releases active ingredient in the environment of the gastrointestinal tract. Example materials include various polymers. It is also envisioned that active ingredient coated lipid and/or wax particulates can be further encapsulated or coated in enteric materials to target the active ingredient to the small intestine and provide stability in high acid aqueous beverages.

Embodiments herein also include a pH release systems for release of active ingredients during ingestion. In these embodiments, the immediate release phase is a liquid and the extended release phase is based on a lipid, wax or lipid/wax particulate. The immediate release phase has a pH modified to be from about 2 to about 5, more particularly from about 3 to about 4, and most typically from about 3.4 to about 3.9. The pH of the liquid immediate release phase is adjusted using citric acid, malic acid, etc. The liquid immediate release phase can include one or more active ingredients as discussed herein, or can simply be a vehicle for flavoring or other non-active ingredient inclusion, i.e., not include an active ingredient. The overall specific gravity of the liquid immediate release phase is typically about 0.8 to 1.2 g/ml, and more typically about 0.9 to 1.1 g/ml. In these embodiments, the extended release phase is composed of lipid, wax or lipid/wax particulates that stay intact at a pH below about 5. Lipid, wax or lipid/wax particulates that stay intact below pH 5 include: stearic acid, stearyl alcohol, candelilla wax, for example.

Where the immediate release phase is an aqueous liquid, and the one or more extended release phases are composed of one or more lipids or wax compositions, the composition often will include a gum system to keep the lipid or wax particles buoyant. Gum systems in accordance with the present disclosure include gellan gum along with ions from sodium citrate and calcium chloride. Gellan gum of particular use include Kelcogel® F (low-acyl gellan gum). In some embodiments the gellan gum can comprise about 0.010% to 0.1% by weight of a composition, and in other embodiments the gellan gum comprises about 0.012% to 0.05% by weight of the composition. The level of composition thickness is determined by the type and amount of gellan gum, and the calcium and sodium ion concentration. Other gum systems can include xanthan gum, guar gum or a combination of gellan gum, xanthan gum and guar gum.

The pH release system is developed to begin release of active ingredients from the lipid, wax or lipid/wax particulates, suspended in liquid, upon a change of pH above about 5 or 6. This is the pH typically associated with the particulates entering the small intestine (typically the pH of the duodenum is about 6 and the terminal ileum is about 7.4). The particulates remain relatively stable at the pH associated with the stomach (below 5, and more typically below 3). As such, the active ingredient is predominately stable and not released until the composition reaches the small intestine, where release of the active begins. In compositions that include an active in the immediate release phase, the active is available as soon as the composition enters the user's mouth.

A multi-phase sports nutritional release composition in accordance with the present invention is a combination of sports nutritional ingredients in an extended release phase, but more typically in an immediate release phase and an extended release phase. A sports nutritional ingredient can be in any one or more of a composition's release phase, e.g., caffeine can be in the immediate release phase as well as the extended release phase, or could be only in the immediate or only in the extended release phases dependent on the target use. In this manner, embodiments of the present disclosure provide significant control over the timing of release of any one active ingredient. Further, multiple extended release phases can be included in any one release composition, for example, a release composition having an immediate release, an extended release that covers up to 90 minutes and an extended release that covers up to 150 minutes, thereby providing overlapping release profiles for active ingredients, all within the same release composition.

In some instances, the ratio of immediate release phase compounds (active ingredient(s)) to extended release compounds (lipid, wax or lipid/wax and active ingredient(s)) is 1:100 to 100:1 based on weight percent, typically 1:50 to 50:1, more typically 1:10 to 10:1 and most typically 1:5 to 5:1. In other instances, the ratio is in the range of 1:1, 1:2 to 2:1, 1:3 to 3:1 and 1:4 to 4:1.

For example, release compositions having a powder immediate release phase could have some instant release aspects and some sustained release aspects dependent of the amount and coating of the lipid particulates in relation to the active ingredient not coated or carried by the lipid and/or wax particulate.

Release compositions composed of a coated lipid particulate in a dry bulk powder immediate release phase are typically sold and stored as such, for example. Prior to ingestion by a user, the release composition can be added to a medium for ingestion by a user, for example, mixed into an aqueous solution or added to a solid food item. Typically, a predetermined amount of release composition is added to the liquid or solid for maximum benefit to the user and consistent with the active ingredient release profile(s).

Release compositions composed of a coated lipid and/or wax particulate in an aqueous liquid immediate release phase are typically sold and stored in a predetermined ingestion size. For example, sold as a 4, 6, 8 or 12 oz. liquid serving. In some embodiments, the coated lipid and/or wax particulate is stored separately from the liquid immediate release phase so as to be added by the user to prepare the release composition just prior to use. In other embodiments, the extended release composition is composed of a coated lipid, wax and/or lipid/wax particulate extended release phase blended/combined into the aqueous immediate release phase. Often, the pre-combined liquid immediate release phase and lipid, wax and/or lipid/wax extended release particulates are combined with sufficient preservatives (sodium benzoate, potassium benzoate, for example) to avoid filtration or heating. These drinks would be prepared using cold fill applications to avoid melting the lipid and/or wax constituents of the composition.

In another embodiment, the release composition is directed toward 1 to 2 oz. liquid servings that would be focused on higher active ingredient concentrations. For example, a 1 oz. serving having both an immediate and extended release phase directed toward energy enhancement, i.e., an energy shot.

Finally, it should be noted that where an immediate release phase is not required, the extended release coated lipid, wax and/or lipid/wax particulates can be sold as stored in water, seltzer water, flavored water, and the like.

Embodiments herein also include methods of preparing a release composition having an immediate release phase and an extended release phase. Methods comprise identifying one or more active ingredients for immediate release as well as one or more active ingredients for extended release in a user. Based on the extended release profile for the one or more active ingredients, one or more coated lipid and/or wax particulates is prepared by choosing an appropriate lipid(s) and wax(es) for the release timing, and combined with the proper amount of the one or more active ingredients to provide a coated lipid and/or wax particulate or matrix. Coated particulates can then be blended (powder) or added (liquid) to the immediate release phase to present a release composition. A user may then ingest the release composition via a liquid or solid delivery. In some methods, the coated particulates are further encapsulated in a polymer or other coating material to avoid dissolution of the lipid and active ingredient prior to ingestion by the user, or to target the extended release phase to the small intestine or colon for release, for example.

The methods herein also include ingestion of a release composition in a user to provide prolonged activity of a predetermined active ingredient(s). Methods provide for release of the active ingredient over 15 minutes, 30 minutes, 45 minutes, 60 minutes, 75 minutes, 90 minutes, 105 minutes, 120 minutes, and the like. Methods also provide for targeting of the active ingredient to various aspects of the gastrointestinal tract in the user, most typically the upper gastrointestinal tract but also the small intestine and colon.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

### Examples

The following examples are provided for illustrative purposes only and are not intended to limit the scope of the invention.

All dissolution testing was performed using 100 ml vessels having small paddles that rotate at 137 RPM. The temperature was maintained at 37.5° C. Approximately 100 ml of media (PBS pH 6.5 with 1.25 wt % or 2.0 wt % NaTC/POPC) was added to each vessel and equilibrating to temperature. To start testing, target release composition was added to vessels while stirring and a timer started. At 15, 30, 60, 90, 240, 360 and 480 minutes, a 1 ml aliquot was removed through a cannula fitted with a 70 μl full flow filter. Removed sample was transferred to a microfuge tube and centrifuged at 21,000 g for 5 minutes. 100 μl of sample supernatant was added to a HPLC vial, and run on HPLC using a 9/1 THF/H₂O. Samples were then tested using a HPLC/CAD Detector Method. All testing was performed on phosphatidic acid as the active ingredient.

### Example 1: Time Release Study On A Lipid Particulate Composition

A time-release dissolution profile was performed on an extended release phase in accordance with an embodiment herein.

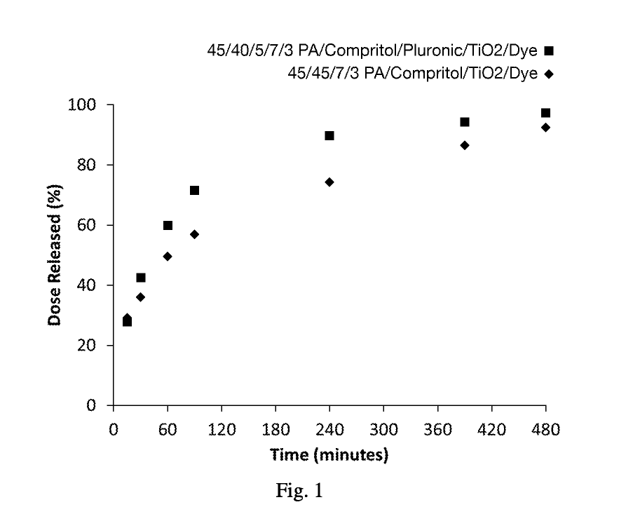

As shown in FIG. 1, the lipid particulate was a combination of 45% by weight of phosphatidic acid, 45% compritol, 7% titanium dioxide, and 3% FD&C Yellow Lake #5. The supplement was tested as discussed above for phosphatidic acid LMP dissolution. As shown by diamonds in FIG. 1, phosphatidic acid was 25% released at 15 minutes, 35% released at 30 minutes, 50% released at 60 minutes, 58% released at 90 minutes, 75% released at 240 minutes, 90% released at 390 minutes and 95% released at 450 minutes. A second profile on FIG. 1 shows a 45% by weight phosphatidic acid, 40% Compritol, 5% Pluronic, 7% titanium dioxide, and 3% FD&C Yellow Lake #5 dye (squares). In this extended release phase the phosphatidic acid was released at 25% released at 15 minutes, 41% at 30 minutes, 60% released at 60 minutes, 75% released at 90 minutes, 90% released at 240 minutes, 95% released at 390 minutes and 98% released at 450 minutes.

The results show a significant extension of the dissolution of the phosphatidic acid from the lipid, thereby providing a significant benefit on the control and design of supplement release to a subject's system. Of particular interest, the lipid particulate including 45% Compritol provided an extended release of the phosphatidic acid as compared to the 40% Compritol and 5% Pluronic.

### Example 2: Time Release Study on a Lipid Particulate Composition

A time-release dissolution profile was conducted on several different extended release compositions in accordance to embodiments described herein. As with Example 1, dissolution testing was performed as described above.

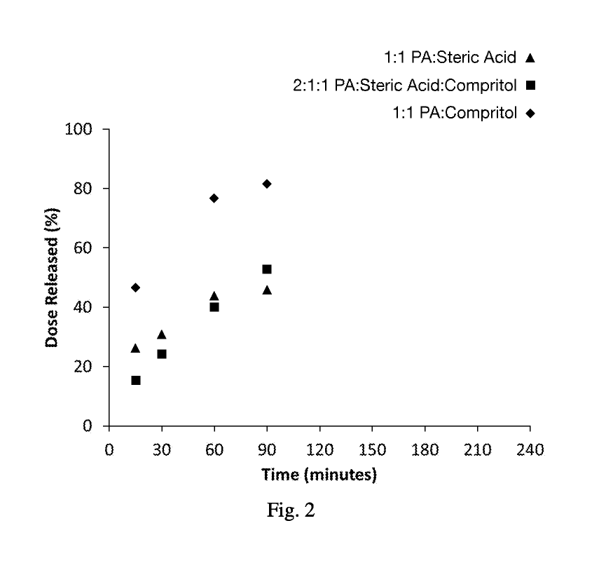

FIG. 2 shows three extended release profiles for several phosphatidic acid—lipid combinations. A first combination of 50% phosphatidic acid coated on 50% by weight steric acid is shown as diamonds. At 15 minutes approximately 45% of the phosphatidic acid was released, at 60 minutes approximately 78% phosphatidic acid was released and at 90 minutes approximately 82% phosphatidic acid was released. A second combination of 50% by weight phosphatidic acid, 25% steric acid by weight and 25% Compritol is shown as squares. In this profile, approximately 17% of the phosphatidic acid was released at 15 minutes, 23% of the phosphatidic acid was released at 30 minutes, 40% of the phosphatidic acid was released at 60 minutes and 50% of the phosphatidic acid was released at 90 minutes. Finally, a 50% by weight phosphatidic acid and 50% by weight Compritol release profile is shown in FIG. 2 as triangles. Here, the phosphatidic acid is approximately 23% released at 15 minutes, 26% released at 30 minutes, 42% released at 60 minutes and 43% released at 90 minutes.

The results show that the combination of Stearic acid and Compritol can be used to significantly modify the release profile of phosphatidic acid over the course of 90 minutes. The data provides further utility for design of an extended release phase with one or more active ingredients for release to a user over a predetermined amount of time.

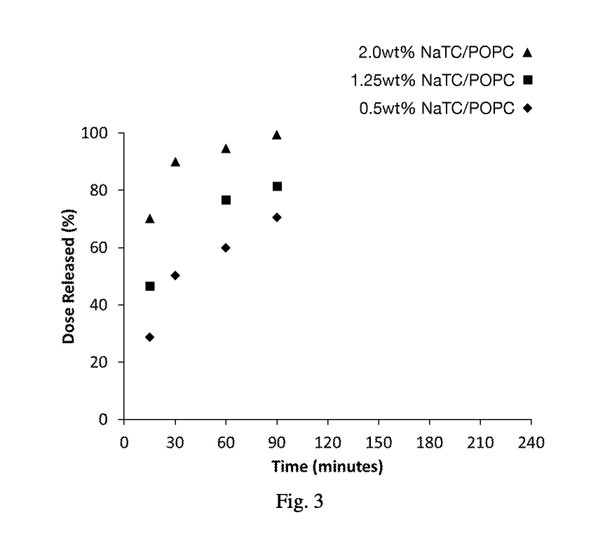

It is also shown that the environment that the lipid particulate is found in also is a factor on the dissolution of a particular extended release phase. In FIG. 3, the same extended release phase (45% phosphatidic acid, 45% Stearic acid, 7% TiO₂ and 3% Dye) was tested using three different dissolution media additions: 2 wt % NaTC/POPC (triangles), 1.25 wt % NaTC/POPC (squares) and 0.5 wt % NaTC/POPC (diamonds). This data shows that the extended release shown in FIGS. 1 and 2 are specific to the lipid and not due to a non-specific effect. The dissolution of the phosphatidic acid is accelerated by increasing amounts of NaTC/POPC.

### Example 3: Time Release Caffeine in a Pre Workout

A time-release composition was prepared using the lipid particulates in Example 2 with a pre-workout powder that includes caffeine, herbal extract, and various vitamins to provide extended energy and focus. Here the caffeine, herbal extract and vitamins would be coated/carried by the lipid or extended phase.

### Example 4: Time Release Pump/Vascularity Product

A time-release norvaline composition was prepared having a two hour release profile so as to behave as an arginase inhibitor. The time release composition was blended with other compounds to promote vasodilation (citrulline derivatives, arginine derivatives, nitrates, etc.) to maintain pump for the duration of the workout.

### Example 5: Time Release Caffeine Product

A caffeine time release composition is provided. The composition is a beverage having a shelf pH of from 1 to 4, although more typically, from 3.0 to 3.5. The beverage is storage stable in the above described pH range. The beverage has a liquid immediate release phase, and an extended release phase, the extended release phase composed of a combination of caffeine, lipid and wax. The extended release phase includes: 10-15% by weight caffeine, 35% by weight candellila wax, 38.34% by weight stearyl alcohol, 15% by weight stearic acid, and 1% by weight titanium dioxide. The immediate release phase may include caffeine as well or have little or no active ingredient.

The pH of the beverage is made through addition of an appropriate amount of citric acid and/or malic acid to put the beverage at a pH of 3.0 to 3.5. Once the beverage is consumed, the extended phase will remain stable until the composition, and more particularly, the extended release phase, reaches a pH closer to, or above, 6. At that pH, the caffeine carried by the lipid and wax particles begins to be released. Note that the above composition may also include a gellan gum to improve suspension of the particulates in the beverage.

### Example 6: Time Release Amino Acid Product

An amino acid time release composition is provided. The composition is a beverage having a shelf pH of from 1 to 4, although more typically, from 3.0 to 3.5. The beverage is storage stable in the above described pH range. The beverage has a liquid immediate release phase, and an extended release phase, the extended release phase composed of a combination of amino acids, lipid and wax. The extended release phase includes: 10-15% by weight amino acid (Leucine, Isoleucine, and/or Valine), 35% by weight candellila wax, 38.34% by weight stearyl alcohol, 15% by weight stearic acid, and 1% by weight titanium dioxide. The immediate release phase may include one or more amino acids as well.

The pH of the beverage is made through addition of an appropriate amount of citric acid and/or malic acid to put the beverage at a pH of 3.0 to 3.5. Once the beverage is consumed, the extended phase will remain stable until the composition, and more particularly, the extended release phase, reaches a pH closer to, or above, 6. At that pH, the amino acid(s) carried by the lipid and wax particles begins to be released. Note that the above composition may also include a gellan gum to improve suspension of the particulates in the beverage.

### Example 7: Composition of Illustrative Product

A 5 calorie per serving time-release drink composed of the ingredients listed in Table 1. The basic formula for one composition embodiment is also provided as: 0-10% actives (more typically 0.5% to 1.5% and most typically around 0.8%), 0.1% preservatives, 0.012% gum system, and 0.58% flavor system. Water is used to Q.S. All percent are in weight percent.

TABLE 1
Illustrative multi-phase sports nutritional beverage:

| | Unit | % Formula | grams/serving |
|---|---|---|---|
| Ingredient (active) | | | |
| caffeine anhydrous | mg | 0.07218 | 0.24107 |
| caffeine beads- | mg | 0.14970 | 0.50000 |
| capsugel | | | |
| niacinamide | mg | 0.00575 | 0.01920 |
| pyridoxine HCL | mg | 0.00389 | 0.01300 |
| methycobalamin | μg | 0.0000097 | 0.0000325 |
| calcium D pantothenic | mg | 0.00392 | 0.01308 |
| acid | | | |
| guarana | mg | 0.00030 | 0.0010 |
| betaine | g | 0.44910 | 1.50000 |
| sensoril | mg | 0.01497 | 0.05000 |
| cognizing citicoline | mg | 0.00030 | 0.00100 |
| theanine | mg | 0.03087 | 0.10309 |
| GABA | mg | 0.07485 | 0.25000 |
| ingredients (inactive) | | | |
| sodium benzoate | mg | 0.05000 | 0.16700 |
| potassium sorbate | mg | 0.05000 | 0.16700 |
| kelcogel F ® (gellan | mg | 0.05200 | 0.17368 |
| gum) | | | |
| sodium citrate | mg | 0.03100 | 0.10354 |
| calcium chloride | mg | 0.03800 | 0.12692 |
| sucralose | mg | 0.05240 | 0.17500 |
| ace-K | mg | 0.02994 | 0.10000 |
| citric acid | mg | 0.02994 | 0.10000 |
| malic acid | mg | 0.11976 | 0.40000 |
| natural bitter blocker | mg | 0.05988 | 0.20000 |
| natural pear flavor- | mg | 0.01198 | 0.04000 |
| prinova | | | |
| n&a blue raspberry | mg | 0.13473 | 0.45000 |
| flavor-prinova | | | |
| natural blueberry | mg | 0.04790 | 0.16000 |
| flavor-prinova | | | |
| natural vanilla flavor- | mg | 0.01796 | 0.06000 |
| prinova | | | |
| blue #1 powder | mg | 0.00030 | 0.00100 |
| water | mg | 98.13904 | 327.78438 |

What is claimed is:

1. A multi-phase release composition comprising:
a liquid immediate release phase; and
a solid extended release phase of an active ingredient carried on or within a particulate, the particular composed of one or more of stearic acid, stearyl alcohol or candelilla wax, or a blend of one or more of stearic acid, stearyl alcohol or candelilla wax; wherein
the immediate release phase has a pH of from about 2 to about 5; and
the solid extended release phase is configured to begin release of the active ingredient at a pH above about 5.

2. The multi-phase release composition of claim 1, wherein the liquid immediate release phase further comprises an active ingredient.

3. The multi-phase release composition of claim 1, wherein the extended release phase is configured to begin release of active ingredient at a pH above 6.

4. The multi-phase release composition of claim 1, wherein the liquid immediate phase further comprises gellan gum, and sodium citrate or calcium chloride.

5. The multi-phase release composition of claim 4, wherein the immediate release phase has a pH of from about 3.5 to about 3.9; and the gellan gum is low-acyl gellan gum.

6. The multi-phase release composition of claim 5, wherein the active ingredient is caffeine.

7. The multi-phase release composition of claim 5, wherein the active ingredient is an amino acid.

8. The multi-phase release composition of claim 5, wherein the immediate release phase has a density of from about 0.95 to 1.05 g/ml.

9. The multi-phase release composition of claim 5, wherein the active ingredient is from about 1 to about 15% by weight of the composition.

10. The multi-phase release composition of claim 9, wherein the gellan gum is about 0.01 to about 0.015% by weight of the composition.

11. A method of making a multi-phase sports nutritional release composition comprising:
capturing one or more target sports nutritional active ingredients on a lipid to form a coated lipid particulate;
obtaining one or more target sports nutritional active ingredients in a dry powder form; and
blending a portion of the coated lipid particulates with a portion of the dry powder to form a blended multi-phase sports nutritional release composition.

12. The method of claim 11, wherein the lipid is selected from the group consisting of stearic acid, Compritol, Pluronic and mixtures thereof.

13. The method of claim 11, wherein the one or more target sports nutritional active ingredients is caffeine.

14. A method of extending an activity of an active ingredient in a user by providing a release composition for ingestion by the user wherein the release composition comprises an immediate release phase and an extended release phase, wherein the active ingredient is in at least the extended release phase.

15. The method of claim 14, wherein the extended release phase comprises a wax and the active ingredient.

16. The method of claim 15, wherein the active ingredient is caffeine.

17. The method of claim 15 wherein the active ingredient is phosphatidic acid.

18. The method of claim 15, wherein the extended release phase further comprises a lipid.

19. The method of claim 18, wherein the wax is a 10 to 30% w/w combination with the lipid.

20. The method of claim 19, wherein the wax is selected from the group consisting of bees wax, rice bran wax, camauba wax, and candelilla wax.
